(19) United States
(12) Patent Application Publication
(10) Pub. No.: US 2026/0083573 A1
(43) Pub. Date: Mar. 26, 2026

(54) SURGICAL SYSTEMS AND METHODS INCLUDING ALIGNING GUIDES FOR PERFORMING A PERCUTANEOUS BUNION CORRECTION

(71) Applicant: Fusion Orthopedics USA, LLC, Mesa, AZ (US)

(72) Inventors: Adam J. Cundiff, Gilbert, AZ (US); Nathan G. Peterson, Gilbert, AZ (US); Eli W. Jacobson, Chandler, AZ (US)

(73) Assignee: Fusion Orthopedics USA, LLC, Mesa, AZ (US)

(21) Appl. No.: 19/409,111

(22) Filed: Dec. 4, 2025

Related U.S. Application Data
(63) Continuation-in-part of application No. 18/774,589, filed on Jul. 16, 2024.

(51) Int. Cl. A61F 2/46; A61B 17/17; A61F 2/42

(52) U.S. Cl. CPC A61F 2/4606; A61B 17/1775; A61F 2/4225; A61F 2002/4677

(57) ABSTRACT

Surgical systems and methods for correcting a bunion percutaneously including aligning guides are provided herein. At least one embodiment includes a handle including a rod with a first end including a first longitudinal axis and a second end including a second longitudinal axis offset from the first longitudinal axis. The handle can couple to a gadget including a leg that couples to the handle, and a translator that extends and retracts the leg. The gadget can also a slide with a guide body to guide a guide wire for accurate placement of a screw. The surgical system can also include a periscope to assist in pre-visualization of the screw placement.

### REFERENCE TO RELATED APPLICATION

This application is a Continuation-In-Part of and claims priority to U.S. patent application Ser. No. 18/774,589, now U.S. Patent No. XX,XXX,XXX, filed on Jul. 16, 2024, the contents of each of which are incorporated herein by reference in their entirety.

### FIELD OF THE TECHNOLOGY

The present technology relates generally to surgical aligning guides, and more particularly to, surgical instruments and systems for a minimally invasive approach to bunion correction.

### BACKGROUND

It is desirable to have a minimally invasive approach to a bunion correction surgery because it minimizes trauma to patient tissues which in turn positively effects patient outcomes. Minimally invasive surgical approaches are difficult because visual access to the surgical site is limited or restricted. In many procedures radiographs are used to visualize steps of the surgical procedure and portions of the patient's anatomy. In a chevron bunion correction, the placement of the bone screws requires a precision that is difficult to achieve with a radiograph. Thus, alignment tools could be helpful.

### SUMMARY

A surgical apparatus that can include a handle, wherein the handle is configured to be couplable to a gadget. The handle can include: a rod including a first end and a second end that is opposite the first end, a first longitudinal axis of the first end, and a second longitudinal axis of the second end, wherein the first longitudinal axis is offset from the second longitudinal axis by an extension. The handle can also include a coupler removably coupled to the rod. The coupler can include a first end and a second end opposite the first end. The handle can include a head that can be coupled to the rod second end. The coupler second end can be coupled to the head. The first and second longitudinal axis can be parallel and an extension length apart, e.g. they are offset by a length of the extension. The extension length can be in the range of 2 mm-50 mm.

The surgical apparatus can also include a gadget. The gadget can include: a body that can include a leg that can be coupled to the handle through the coupler; a slide that can be slidably coupled to the body; a guide body that can be slidably coupled to the body and configure to hold a guide wire, and is configured to be rotatable. The slide can include a lock to prevent the slide from moving from a desired position. The lock can have a handle.

The surgical apparatus can also include a translator. The translator can be coupled to the leg of the body and it can be configured to extend and retract the leg through the activation of an activation mechanism. The activation mechanism can be a dial. The dial can be coupled to a threaded rod which is in turn coupled to a set of threads included on/in the leg. The turning of the dial turns the rod and the rod threads, which extends and retracts the leg through the leg threads.

The surgical apparatus can also include a radiograph positioning tool that can assist a user in determining if the gadget is in a correct position.

The surgical apparatus can also include a guide wire sheath coupled to the body and configured to guide a k-wire to a desired position.

The surgical apparatus can include a rotation arm. The rotation arm can be removably couplable to the body. The rotation arm is configured to hold a k-wire at angles that are in approximately 5° increments. It is also configured to assist in the rotation of a metatarsal capital fragment.

The surgical apparatus can include at least one radiograph positioning tool. The radiograph positioning tool can be two bars that are configured to align when viewed in a radiograph taken in the transverse plane.

The surgical apparatus can include a periscope. The periscope can be couplable to the guide body, or it can be integral with the guide body. The periscope can hold a sight wire (k-wire) in the same orientation that a guidewire sheath will guide a k-wire, which can in turn guide at least one screw into at least one target bone and/or into a proximal portion of a metatarsal and a metatarsal capital fragment. The periscope can include a radiograph positioning tool.

A method of performing a bone alignment correction can include the following. A handle can be coupled to a first bone portion by inserting a rod first end into the first bone portion, a rod second end can be opposite the rod first end and can be coupled to a head. The head can be coupled to a coupler which can have a first end and a second end opposite the first end. The coupler second end can be coupled to the head. The coupler first end can be coupled to a gadget leg, thereby coupling the gadget to the handle. The gadget can also include a body. The body can include at least one aperture. A k-wire can be inserted through the aperture into a bone of a subject to secure the gadget. The gadget can include a slide which can be extended. The gadget can include a periscope which can be coupled to the guide body. The periscope can include a sight wire which can be inserted into at least one periscope sight aperture. The position of the sight wire can be checked radiographically. If the position of the sight wire is not a desired position, the slide and guide body positions can be adjusted to achieve a desired sight wire position. Guide wire(s) can be inserted through the guide wire sheath and into the foot of a subject to hold a desired position of the bones or bone portions. Screws can be inserted in the foot of a subject over the guide wire(s).

### BRIEF DESCRIPTION OF THE DRAWINGS

To readily understand the advantages and benefits of the technology, a more particular description of the technology briefly described above will be rendered by reference to specific embodiments that are illustrated in the appended drawings. Understanding that these drawings depict typical embodiments of the technology, and are therefore not to be considered to be limiting of its scope, the technology will be described and explained with additional specificity and detail through the use of the accompanying drawings, in which:

FIG. 1 is a schematic diagram illustrating an embodiment of a surgical system including a handle including a coupler in a first position;

FIG. 2 is a schematic diagram illustrating the surgical system of FIG. 1 with the coupler in a second position;

FIG. 3 is a schematic diagram illustrating an isometric view of the surgical system of FIG. 1 with the coupler removed;

FIG. 4 is a schematic diagram illustrating an isometric view of various embodiments of a rod first end of the surgical system of FIG. 1;

FIGS. 5 and 6 are schematic diagrams illustrating isometric views of an embodiment of a coupler included in various embodiments of the surgical system of FIG. 1;

FIGS. 7 and 8 are schematic diagrams illustrating various embodiments of the surgical system including a gadget and a handle;

FIG. 9 is a schematic diagram illustrating a top view of an embodiment of a surgical system including a gadget;

FIG. 10 is a schematic diagram illustrating an isometric view of an embodiment of the surgical system of FIG. 9 including a bottom of a gadget;

FIG. 11 is a schematic diagram illustrating a top view of an embodiment of the surgical system of FIG. 9 including a slide and a radiograph positioning tool;

FIGS. 12 and 13 are schematic diagrams illustrating lateral views of various embodiments of the surgical system of FIG. 9 including a radiograph positioning tool;

FIG. 14 is a schematic diagram illustrating an isometric view of the surgical system of FIG. 9 including a closed slide;

FIG. 15 is a schematic diagram illustrating an isometric view of the surgical system of FIG. 9 including a fully extended slide;

FIG. 16 is a schematic diagram illustrating a top view of the surgical system of FIG. 9 including a closed slide;

FIG. 17 is a schematic diagram illustrating a top view of the surgical system of FIG. 9 including a fully extended slide;

FIG. 18 is a schematic diagram illustrating an isometric view of the surgical system of FIG. 9 including a guide body;

FIG. 19 is a schematic diagram illustrating a top view of the surgical system of FIG. 9 including a rotatable guide body;

FIG. 20 is a schematic diagram illustrating an isometric view of an embodiment of the surgical system including a drill sleeve;

FIG. 21 is a schematic diagram illustrating a side view of the surgical system of FIG. 20;

FIG. 22 is a schematic diagram illustrating an end view of an embodiment of the surgical system of FIG. 20;

FIG. 23 is a schematic diagram illustrating an isometric view of an embodiment of the surgical system including a guide wire sheath;

FIG. 24 is a schematic diagram illustrating a top view of an embodiment of the surgical system including two drill sleeves and guide wire sheaths;

FIG. 25 is a schematic diagram illustrating an isometric view of the surgical system of FIG. 19 including at least one drill sleeve and guide wire sheath;

FIG. 26 is a schematic diagram illustrating an isometric view of an embodiment of the surgical system including a periscope;

FIG. 27 is a schematic diagram illustrating an embodiment of the surgical system including a periscope;

FIG. 28 is a schematic diagram illustrating an isometric view of the surgical system of FIG. 27;

FIG. 29 is a schematic diagram illustrating an isometric view of an embodiment of the surgical system including a leg;

FIG. 30 is a schematic diagram illustrating a top view of an embodiment of the surgical system including a translator;

FIG. 31 is a schematic diagram illustrating an isometric view of an embodiment of the surgical system including a rotation arm;

FIG. 32 is a schematic diagram illustrating an isometric view of an embodiment of the surgical system including a rotation arm;

FIG. 33 is a schematic diagram illustrating an isometric view of an embodiment of the surgical system including a rotation arm;

FIG. 34 is a schematic diagram illustrating a side view of an embodiment of the surgical system including a screw;

FIG. 35 is a schematic diagram illustrating an isometric view of the surgical system of FIG. 34;

FIG. 36 is a schematic diagram illustrating an end view of the surgical system of FIG. 34;

FIG. 37 is of a schematic diagram illustrating the surgical system of FIG. 1 with the handle inserted into the bone of a subject;

FIG. 38 is a schematic diagram illustrating the surgical system of FIG. 1 coupled to the gadget of FIG. 9;

FIG. 39 is a schematic diagram illustrating the surgical system of FIG. 1 coupled to the gadget of FIG. 9 including an attachment mechanism;

FIG. 40 is a schematic diagram illustrating the surgical system of FIG. 1 coupled to the gadget of FIG. 9 including an open slide;

FIG. 41 is a schematic diagram illustrating the surgical system of FIG. 1 coupled to the gadget of FIG. 9 including a radiograph positioning tool;

FIG. 42 is a schematic diagram illustrating the surgical system of FIG. 1 coupled to the gadget of FIG. 9 including a rotation arm;

FIG. 43 is an isometric view of a schematic diagram illustrating the surgical system of FIG. 42 including a k-wire;

FIG. 44 is an isometric view of a schematic diagram illustrating the surgical system of FIG. 43 including a translated capital fragment;

FIG. 45 is a schematic diagram illustrating a top view the surgical system of FIG. 44;

FIG. 46 is a schematic diagram illustrating the surgical system of FIG. 45 including a periscope;

FIG. 47 is a schematic diagram illustrating of the surgical system of FIG. 46 including a drill sleeve and a guide wire sheath;

FIG. 48 is a schematic diagram illustrating the surgical system of FIG. 47 without a guide wire sheath;

FIG. 49 is a schematic diagram illustrating the surgical system of FIG. 48 with screws;

FIG. 50 is a schematic diagrams illustrating a dorsal view the surgical system of FIG. 48 with the gadget and handle removed;

FIG. 51 is a schematic diagrams illustrating a plantar view the surgical system of FIG. 50;

FIG. 52 is flowchart illustrating an embodiment of a method of using the surgical system;

FIG. 53 is a schematic diagram illustrating an isometric top view of an embodiment of the surgical system with a plate buffer and open slot;

FIG. 54 is a schematic diagram illustrating an isometric bottom view of FIG. 53;

FIG. 55 is a schematic diagram illustrating the leg of FIG. 53

FIG. 56 is a schematic diagram illustrating a side view of the leg of FIG. 53;

FIG. 57 is a schematic diagram illustrating an axial view of the open slot of FIG. 53;

FIG. 58 is a schematic diagram illustrating a plate buffer of FIG. 53;

FIG. 59 is a schematic diagram illustrating a front view of the plate buffer of FIG. 53;

FIG. 60A is a schematic diagram illustrating a side view of an embodiment of the surgical system including a cuff buffer;

FIG. 60B is a schematic diagram illustrating a top view of FIG. 60A;

FIG. 61A is a schematic diagram illustrating an isometric back view of the surgical system including an embodiment of a periscope including a sheath;

FIG. 61B is a schematic diagram illustrating an isometric back view of FIG. 61A;

FIG. 61C is a schematic diagram illustrating a back view of FIG. 61A;

FIG. 62A is a schematic diagram illustrating an isometric back view of the surgical system including an embodiment of a periscope;

FIG. 62B is a schematic diagram illustrating an isometric back view of FIG. 62A;

FIG. 63A is a schematic diagram illustrating an isometric front view of the surgical system including an embodiment of a periscope including two sight apertures;

FIG. 63B is a schematic diagram illustrating an back view of FIG. 63A;

FIG. 63C is a schematic diagram illustrating a isometric back view of FIG. 63A;

FIG. 64A is a schematic diagram illustrating a side view of the surgical system including an embodiment of a handle;

FIG. 64B is a schematic diagram illustrating an isometric view of FIG. 64A;

FIG. 65A is a schematic diagram illustrating an isometric view of the surgical system including an embodiment of FIG. 63A including a coupler;

FIG. 65B is a schematic diagram illustrating an isometric view of FIG. 65A;

FIG. 66A is a schematic diagram illustrating an isometric view of FIG. 65A including some internal features;

FIG. 66B is a schematic diagram illustrating an isometric view of FIG. 65A including some internal features; and

FIGS. 67A and 67B are schematic diagrams illustrating isometric views of an embodiment of a coupler included in various embodiments of the surgical system of FIG. 64A.

### DETAILED DESCRIPTION OF THE DRAWINGS

It should be understood that the language used in the present disclosure has been principally selected for readability and instructional purposes, and not to limit the scope of the subject matter disclosed herein in any manner. Further, reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including, but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

In addition, as used herein, the term “first bone” can refer to a bone or bone portion. The term “second bone” can refer to a bone that is a different bone than the first bone, or it can refer to a different portion of the same bone as the first bone.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. Aspects of the embodiments are described below with reference to schematic flowchart diagrams and/or schematic block diagrams of methods, apparatuses, and systems according to embodiments. The schematic flowchart diagrams and/or schematic block diagrams in the Figures illustrate the structure, functionality, and operation of possible implementations of apparatuses, systems, and methods according to various embodiments.

It should also be noted that, in some alternative implementations, the functions noted in the block may occur out of the order noted in the Figures. For example, two blocks shown in succession may, in fact, be executed substantially concurrently, or the blocks may sometimes be executed in the reverse order. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment.

The present technology may include any type of surgical system and is not limited to the style of surgical system depicted in the drawings. Furthermore, the described features, structures, or characteristics of the various embodiments may be combined in any suitable manner. One skilled in the relevant art will recognize, however, that embodiments may be practiced without one or more of the specific details, or with other methods, components, materials, and so forth. In other instances, well-known structures, and/or materials are not shown or described in detail to avoid obscuring aspects of an embodiment.

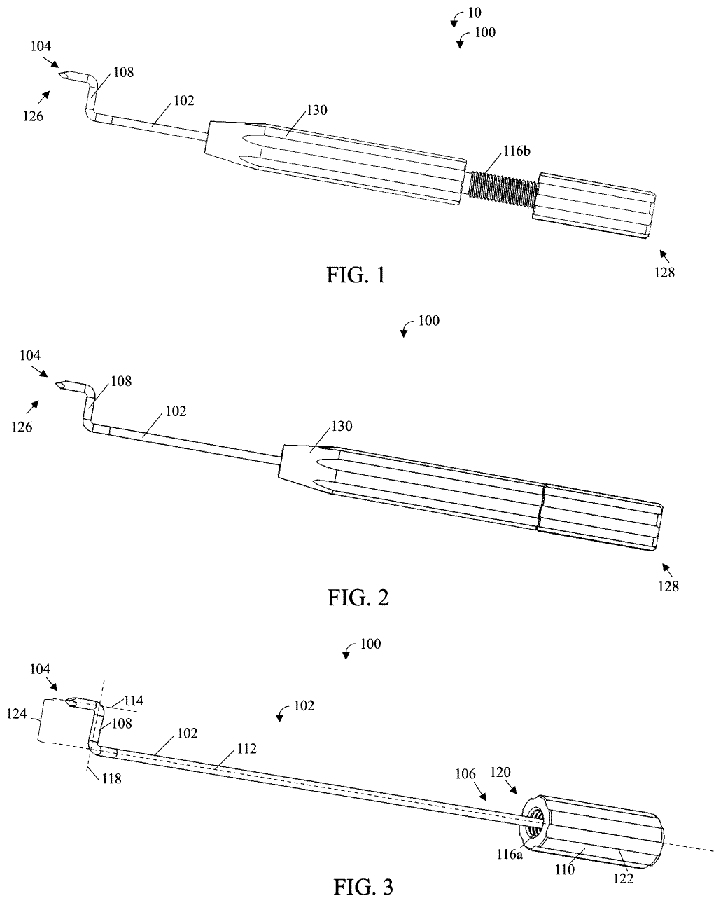

Turning now to the Figures, FIGS. 1 through 67B are schematic diagrams illustrating various views and/or embodiments of a surgical system 10. In various embodiments, the surgical system 10 can be utilized to perform a bone position correction procedure, such as a minimally invasive bunion correction procedure, or tailor's bunion correction procedure, among other bone alignment correction procedures that are possible, each of which is contemplated herein.

The surgical system 10 may be constructed of any suitable material capable of forming the various embodiments of the surgical system 10 described herein. In various embodiments, the surgical system 10 is constructed of a material that can be sterilized, and/or a material that is sterilized. In some embodiments, the surgical system 10 includes stainless steel, radiopaque, titanium, titanium alloy, plastic(s), carbon fibers, and/or aluminum, among other suitable materials that are possible, each of which is contemplated herein. In additional or alternative embodiments, the surgical system 10 includes surgical grade stainless steel, among other suitable surgical grade materials that are possible, each of which is contemplated herein.

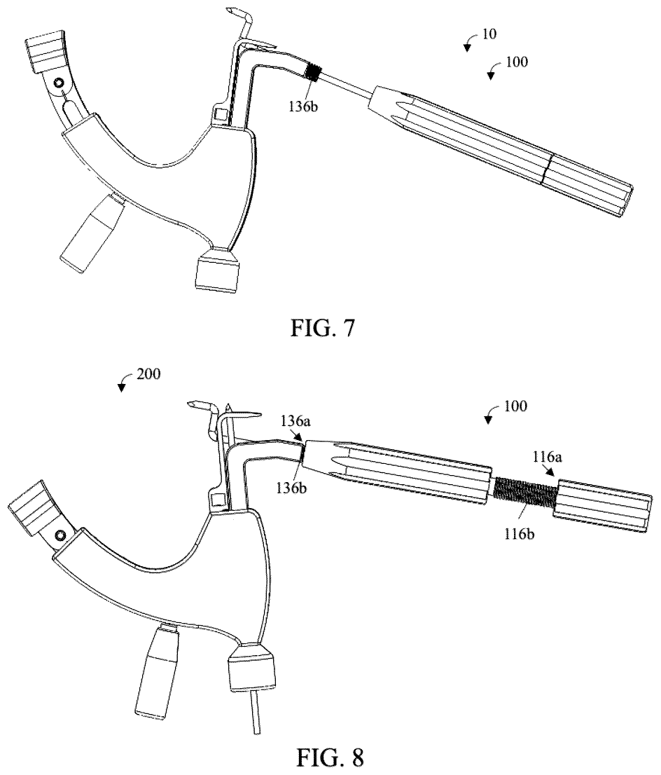

Referring now to the figures, at least in the illustrated embodiment, the surgical system 10 can include, among other components, a handle 100. The handle 100, in various embodiments, can include a rod 102, among other components (e.g., see FIGS. 1-8).

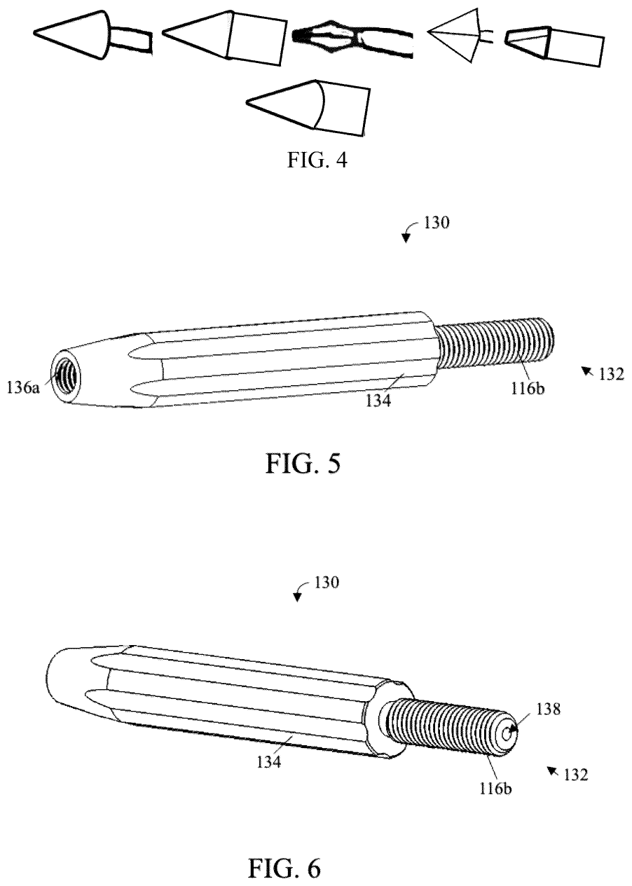

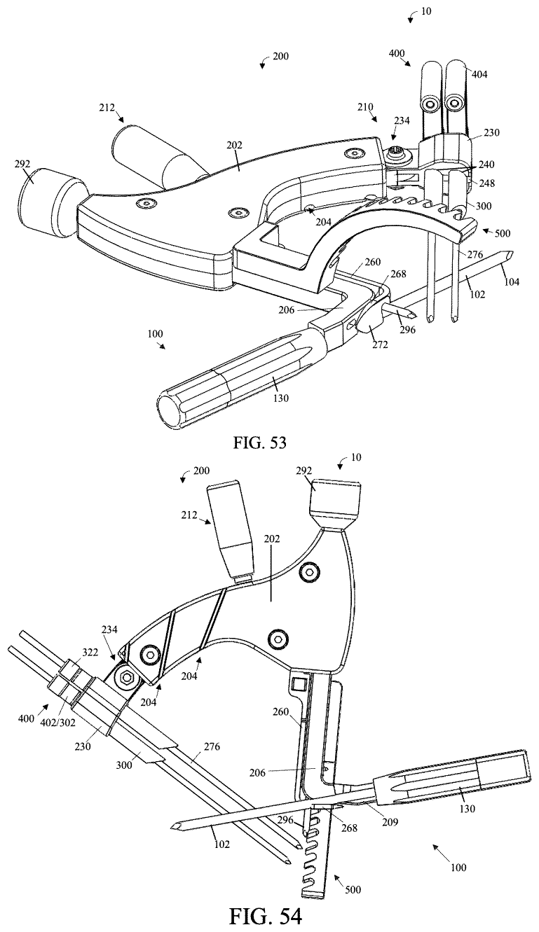

The rod 102, in various embodiments, can include a first end 104 and a second end 106 (e.g., see FIG. 3). The first end 104 can include, among other characteristics, a flat, blunt, wedged, conical, rounded, pointed, and/or spiked contour (e.g., see FIG. 4) among other contours, characteristics, and/or shapes that are possible, each of which is contemplated herein. In some embodiments the rod 102 can be straight (e.g., see FIG. 53). The rod first end 104, in various embodiments, can be coupled to the rod second end 106, or they can be integral.

In some embodiments, the rod 102 can include an extension 108. The extension 108, in various embodiments, can be coupled to the rod first end 104 and/or to the rod second end 106, or they can be integral. The extension 108 can have a longitudinal axis 118 that is not coextensive with the longitudinal axis 112 of the rod second end 106. The extension 108, in at least some embodiments, can have a longitudinal axis 118 that is coextensive with the longitudinal axis 112 of the rod second end 106 (e.g., the rod is straight).

The rod extension longitudinal axis 118, in various embodiments, can be angled relative to the longitudinal axis 112 of the rod second end 106. In some embodiments, the rod extension108 longitudinal axis 118 can be perpendicular to the longitudinal axis 112 of the rod second end 106.

In additional or alternative embodiments, the rod extension 108 longitudinal axis 118 can be angled relative to the longitudinal axis 114 of the rod first end 104. The rod extension 108 longitudinal axis 118 can be perpendicular to the longitudinal axis 114 of the rod first end 104. In some embodiments, the longitudinal axis 114 of the rod first end 104 can be angled relative to the longitudinal axis 112 of the rod second end 106. The longitudinal axis 114 of the rod first end 104 can be parallel relative to the longitudinal axis 112 of the rod second end 106.

The extension 108, in various embodiments, can include a length 124 in the range of about two millimeters to about fifty millimeters (2 mm-50 mm), inclusive, among other lengths that are less than 2 mm or greater than 50 mm that is/are possible, each of which is contemplated herein (e.g., see FIG. 3). In some embodiments, the length 124 is in the range of about five millimeters to about thirty millimeters (5 mm-30 mm), inclusive, among other lengths that are less than 5 mm or greater than 30 mm that is/are possible, each of which is contemplated herein.

In various embodiments, the handle 100 can include a head 110 coupled to the rod 102. In some embodiments the head 110 is attached and/or coupled to the rod second end 106. The head 110, in at least some embodiments, can include an attachment mechanism 116a at a first end 120 of the head 110.

The attachment mechanism 116a can include any suitable attachment mechanism. Examples of an attachment mechanism 116a can include, but are not limited to, one or more threads (e.g., FIG. 3), one or more: complimentary locking contours, apertures, rods, hooks, barbs, screws, locking screws, nuts, bolts, push screws, clamps, spring buttons, ball, retention bearing, and/or groove, etc., among other possible means of attachment, each of which is contemplated herein.

The head 110 can include a grip 122 on at least one exterior surface. The grip 122 can be a contour, a high friction surface, a high friction material, a knurling, and/or one or more grooves, etc., among other possible surfaces and/or materials that include(s) a high or relatively high coefficient of friction, each of which is contemplated herein.

The handle 100, in various embodiments, can include a coupler 130. In some embodiments, the coupler 130 can be removably coupled to the head 110. The coupler can be in a first position (see, e.g., FIG. 1), a second position (see, e.g., FIG. 2), a position in between the first and second position. The coupler 130 can include an attachment mechanism 116b configured to couple the coupler 130 to the head 110.

The attachment mechanism 116b can include any suitable attachment mechanism. Examples of an attachment mechanism 116b can include, but are not limited to, one or more threads, one or more: complimentary locking contours, apertures, rods, hooks, barbs, screws, locking screws, nuts, bolts, push screws, clamps, spring buttons, ball, retention bearing, and/or groove,, etc., among other possible means of attachment, each of which is contemplated herein.

In various embodiments, the coupler 130 can include a grip 134 on at least one exterior surface. The grip 134 can be at least one contour, high friction surface, high friction material, among other possible surfaces and/or materials that include(s) a high or relatively high coefficient of friction, each of which is contemplated herein.

The coupler 130 can include a second attachment mechanism 136a, which can include but are not limited to, one or more threads, one or more: complimentary locking contours, apertures, rods, hooks, barbs, screws, locking screws, nuts, bolts, push screws, clamps, spring buttons, ball, retention bearing, and/or groove, etc., among other possible means of attachment, each of which is contemplated herein. The coupler 130, in various embodiments, can be cannulated 138 and/or hollow.

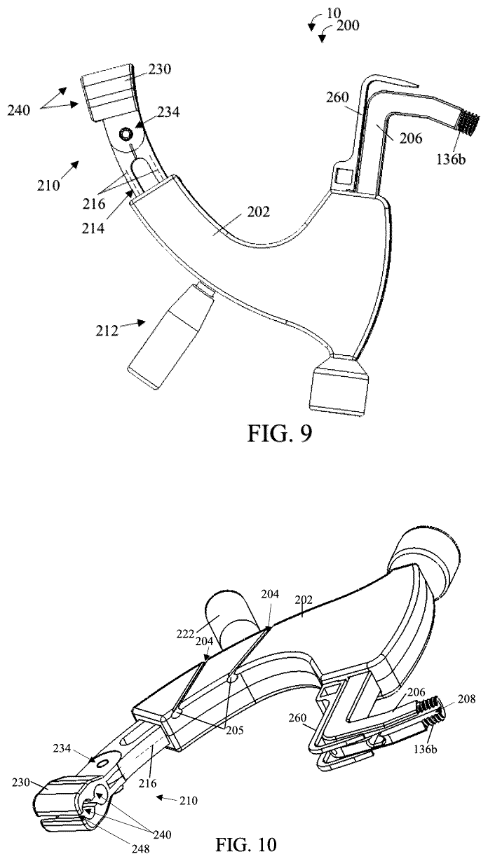

Referring now to FIGS. 8 through 18, the surgical system 10, at least in the illustrated embodiments, can include a gadget 200. The gadget 200 can include a body 202.

The body 202 can include at least one body attachment mechanism 204. The body attachment mechanism 204 can include one or more threads, one or more complimentary locking contours, an aperture, a rod, and/or a hook, etc. among other possible means of attaching the body 202 to the bone(s) of a subject, and/or other surgical device(s), each of which is contemplated herein. In one or more embodiments the attachment mechanism 204 can include a body aperture 205 (see, e.g., FIG. 10). In at least one embodiment the attachment mechanism can include a k-wire 708.

In various embodiments, the gadget 200 can include a leg 206 coupled to the body 202 and attachable to the coupler 130. The leg 206 can include an attachment mechanism 136b, which can include one or more threads, but are not limited to, one or more threads, one or more: complimentary locking contours, apertures, rods, hooks, barbs, screws, locking screws, nuts, bolts, push screws, clamps, spring buttons, ball, retention bearing, and/or groove, etc., among other possible means of attachment, each of which is contemplated herein. Attachment mechanism 136b can be complementary to attachment mechanism 136a. In some embodiments, the leg 206 can include a slot 208 that can facilitate the coupling of the gadget 200 to the handle 100. The slot 208 can be a passageway with two open ends and one open side (e.g., top, bottom, side).

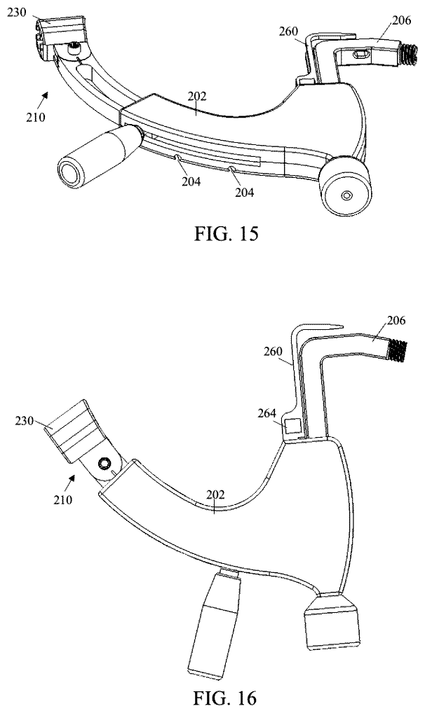

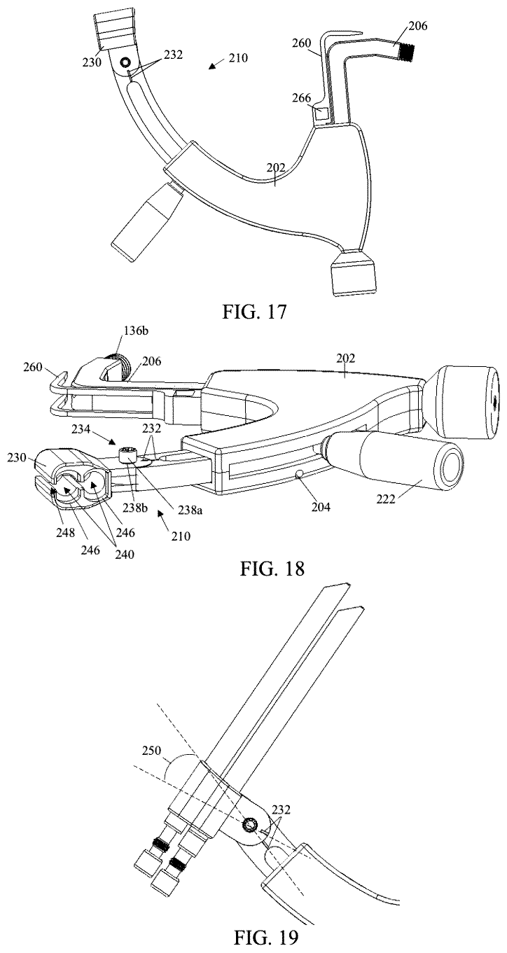

The gadget 200, in some embodiments, can include a slide 210 slidably coupled to the body 202. The slide 210 can be in a first closed position (e.g., see FIGS. 14 and 16), a second fully extended position (e.g., see FIGS. 15 and 17), or a third open position which can be any position in between the first closed position and the second fully extended position, (see, e.g., FIG. 10).

In various embodiments, the slide 210 can include a slide lock 212 coupled to the slide 210 and configured to hold the slide 210 in a desired position relative to the body 202. The lock 212, in various embodiments, can include a pressure fit, a post, an aperture, a screw, and/or a friction lock, etc., or other means of locking the slide in place relative to the body 202, each of which is contemplated herein.

At least in the illustrated embodiment, the slide lock 212 can include an aperture 218 that can be threaded, and a post 220 that can be threaded and complimentary to the aperture 218. In some embodiments, the lock 212 can include a lock handle 222 that can be coupled to the lock post 220.

In at least one embodiment, the lock handle 222 can be removable from (e.g., screwdriver) or integral with the lock post 220. In some embodiments, the lock post 220 forms at least a portion of a screw, or a bolt, among other locking mechanisms, each of which is contemplated herein. In various embodiments, the lock handle 222 can form at least a portion of a screwdriver, drill, etc., among other tools that create torque, each of which is contemplated herein.

In certain embodiments, the slide 210 can include a radiograph positioning tool 214. The radiograph positioning tool 214 can include at least one of an aperture, a bar, text, and/or emoji, etc., among other possible tools, each of which is contemplated herein. At least in the pictured embodiment, the radiograph positioning tool 214 includes two bars 216 within the slide 210 at the same depth (e.g., on the same plane), but near opposing sides. When the slide 210 is on a lateral radiograph, the two bars 216 can appear to align (because both bars 216 are positioned at the same depth and/or on the same plane in the slide 210) (see, e.g., FIG. 12). The appearance of one bar 216 on the radiograph can indicate that the gadget 200 is properly aligned with the foot of a subject. In many embodiments the appearance of one bar 216 aligned with the longitudinal axis of the metatarsal of a patient indicates proper alignment of the gadget 200.

The gadget 200, in various embodiments, can include a guide body 230. The guide body 230 can be coupled to the slide 210 at an angle 250 (see, e.g., FIG. 19).

In certain embodiments, the guide body 230 can include an alignment tool 232 that indicates the guide body 230 is in the default position (see, e.g., FIG. 17) relative to the slide 210, where angle 250 is at a default angle (e.g., 0 degrees, or parallel). The guide body 230 can be rotated to a second position where angle 250 is not zero degrees (0°) (see, e.g., FIG. 19).

In some embodiments, the guide body 230 can include a securing device 234 that locks the guide body 230 into place to prevent the angle 250 from changing when the securing device 234 is engaged (see, e.g., FIG. 18). The securing device 234, in various embodiments, can include least one of an aperture, a screw, a bolt, and/or a nut, etc., among other securing means, each of which is contemplated herein. In some embodiments the securing device 234 is a screw 238a and threaded aperture 238b.

In certain embodiments, the guide body 230 can include at least one sleeve aperture 240 configured to hold a drill sleeve 300, as described in greater detail below. At least a portion of the sleeve aperture 240 can include any suitable shape, including, but not limited to, circular, flat-circle (circle with at least one flat side), elliptical, flat elliptical (ellipse with at least one flat side), oblong, square, rectangular, star, polygonal, and/or hexagonal, among other shapes that are possible, each of which is contemplated herein.

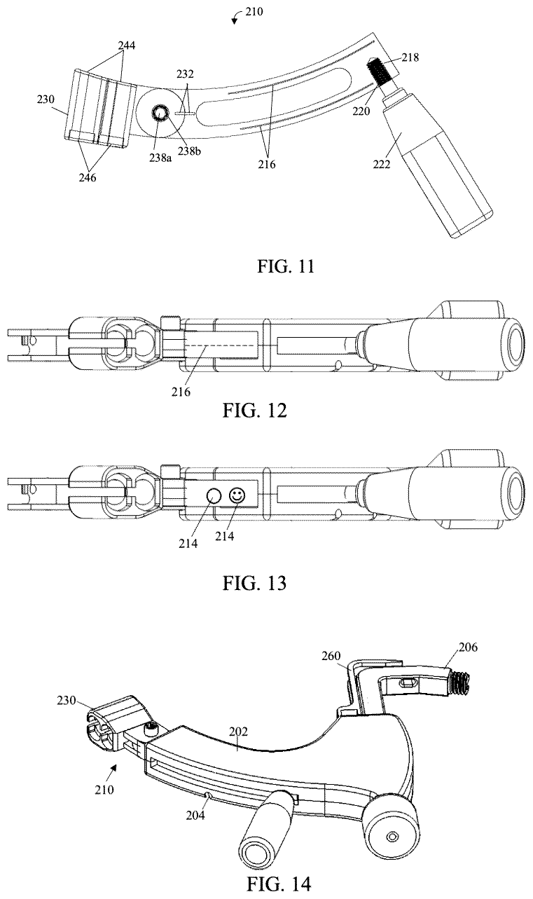

At least in the illustrated embodiment, the sleeve aperture 240 can include at least two shapes, a circular shape near the first side 244 and a non-circular shape at the second side 246 (see, e.g., FIG. 11), for example, flat-circle (circle with at least one flat side), elliptical, flat elliptical (ellipse with at least one flat side), oblong, square, rectangular, star, polygonal, and/or hexagonal, among other suitable shapes and/or pairs of shapes that are possible, each of which is contemplated herein. In some embodiments, the non-circular shape can assist in preventing the sleeve 300 from rotating.

The guide body 230 can also include an opening 248 to assist in the removal of the gadget 200 from the foot of a patient without removing the k-wires. Removing the gadget 200 can give easier access to the k-wires for screw placement if not using the gadget 200 for screw placement.

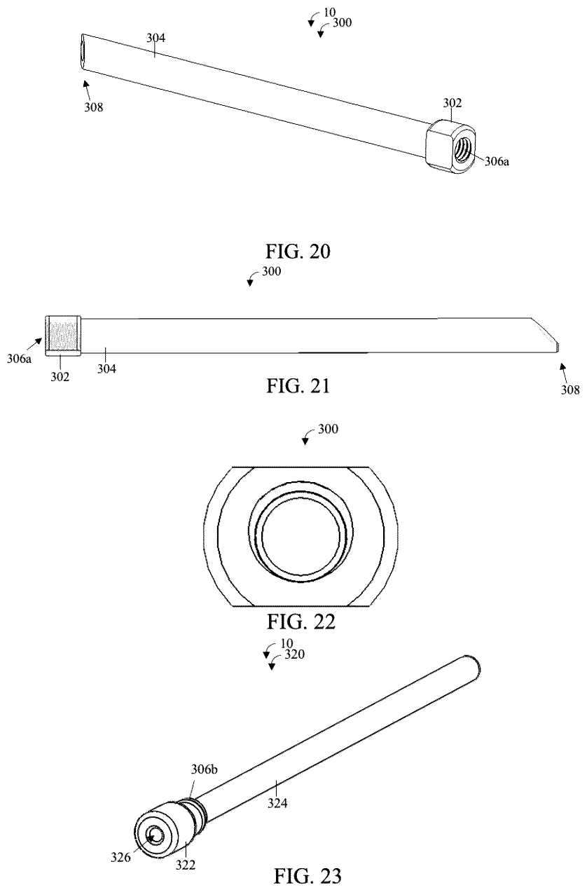

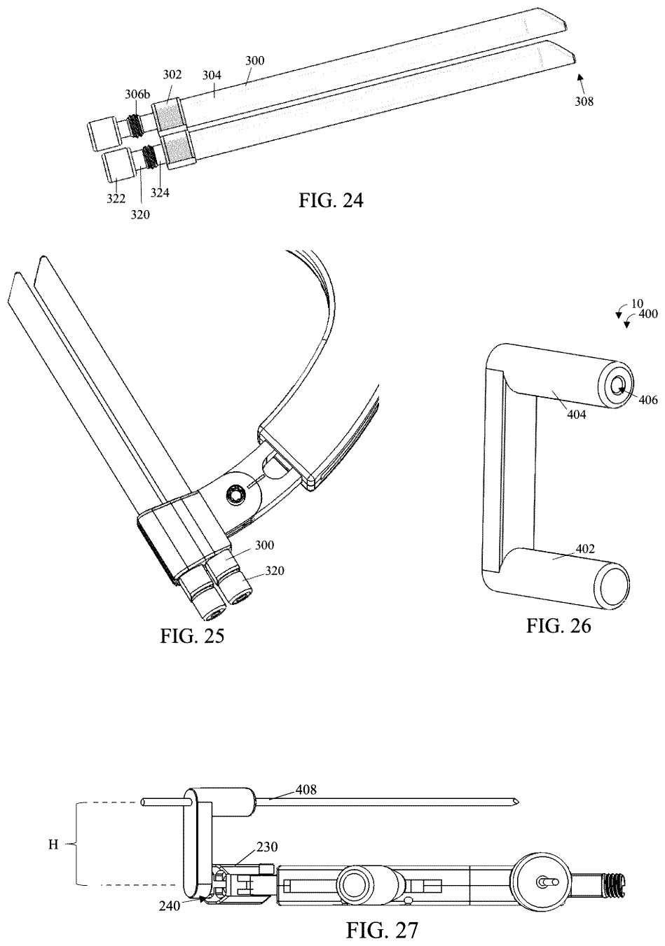

Now referring to FIGS. 19 through 25, the surgical system 10, in some embodiments, can include a drill sleeve 300. The drill sleeve 300 can include a head 302 and a shaft 304.

In some embodiments, the head 302 can include the same contours and/or geometry as the shaft 304. In other embodiments, the head 302 can have one or more different contours than the shaft 304. In at least the illustrated embodiment, the head 302 can include a non-circular contour, and the shaft 304 can include a circular contour. In at least one embodiment the exterior of the shaft 304 can have one or more different contours than the interior of the shaft.

The shaft 304, in various embodiments, can include complimentary contours to the sleeve aperture first side 244. In additional or alternative embodiments, the head 302 can include complimentary contours to the sleeve aperture second side 246. In some embodiments, the shaft 304 can be any length that places the end 308 in contact with the foot of a patient. In some embodiments, the shaft can be any length that places the end 308 in proximity to the foot of a patient.

In some embodiments, the head 302 can include a coupling device 306a to removably couple the guide wire sheath 320 (see below) to the drill sleeve 300. The drill sleeve 300 can include a first end 308 that can be straight (e.g., a blunt cut) or not straight. In some embodiments the drill sleeve first end 308 can be chamfered and/or beveled. In other embodiments, the drill sleeve first end 308 can be configured to conform with human anatomy.

In various embodiments, the surgical system 10 can include a guide wire sheath 320. The guide wire sheath 320 can include, among other features/components, a sheath head 322, a sheath shaft 324, and a coupling device 306b that is complimentary to the coupling device 306a. The coupling device(s) 306a and/or 306b can include one or more threads, one or more complimentary locking contours, apertures, rods, hooks, barbs, screws, locking screws, nuts, bolts, push screws, clamps, spring buttons, ball, retention bearing, and/or groove, etc., among other possible means of coupling the guide wire sheath 320 to the drill sleeve 300, each of which is contemplated herein. The guide wire sheath 320, is cannulated 326 to allow for a k-wire (e.g., a guide wire 276) to be inserted therethrough.

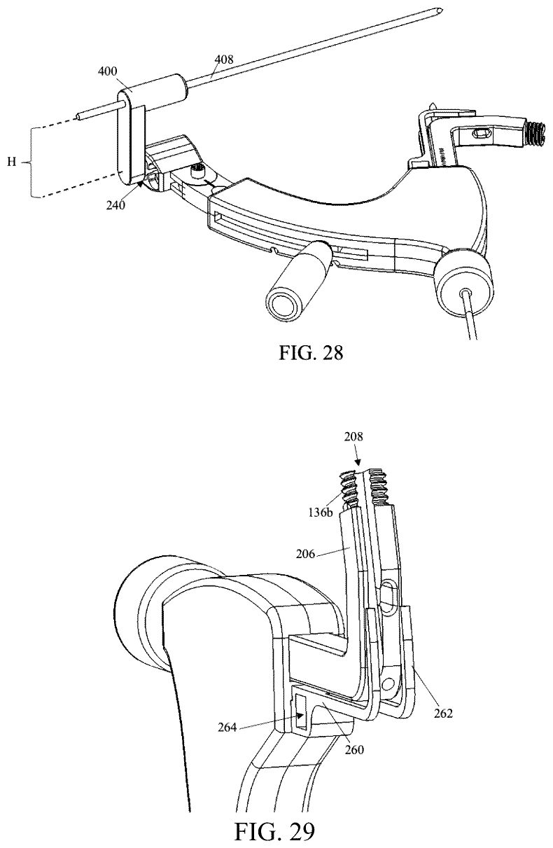

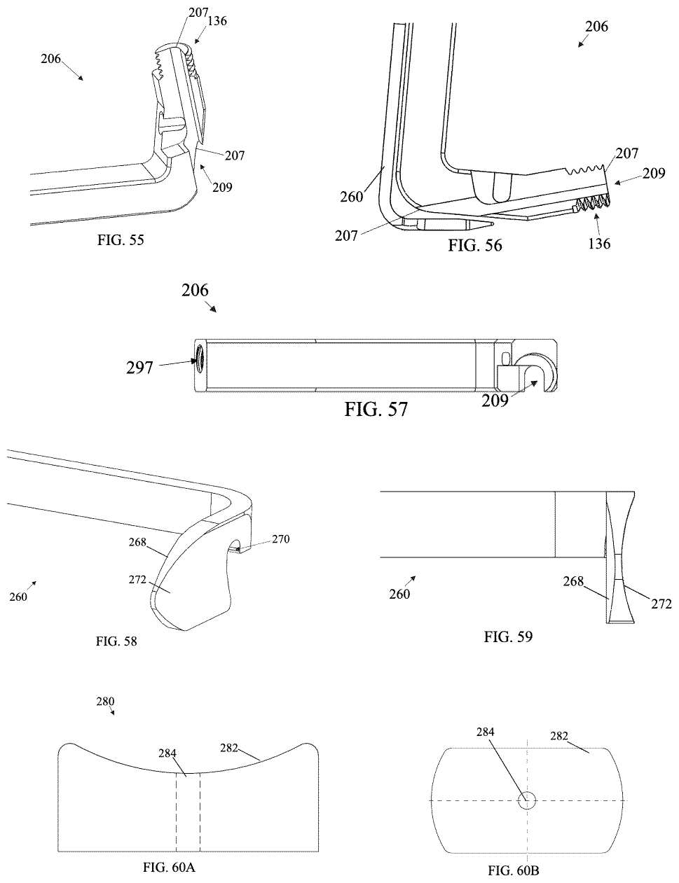

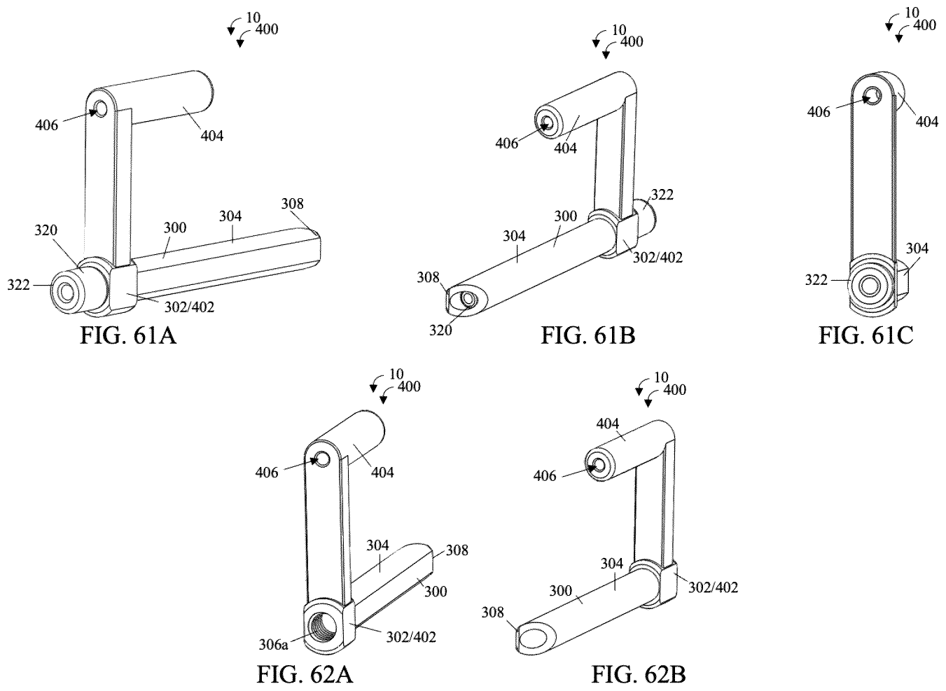

In various embodiments, the surgical system 10 can include at least one periscope 400. In some embodiments, the periscope 400 can include a post 402 that can be inserted into the sleeve aperture 240 to couple the periscope 400 to the guide body 230 (e.g., see FIGS. 26-28, and 61A-62B). The post 402, in various embodiments, can be configured to conform to the sleeve aperture 240. In one or more embodiments, the periscope 400 can be integral with the guide body 230. In certain embodiments, the periscope 400 can be integral with the drill sleeve 300. In further embodiments the periscope 400 can be couplable with the drill sleeve 300. In one or more embodiments the periscope 400 can be integral with the drill sleeve 300 (e.g., see FIGS. 61A-62B). In various embodiments the periscope 400 can be couplable with the drill sleeve 300.

In certain embodiments, the periscope 400 can include at least one sight 404 including at least one sight aperture 406 in which the center of the sight aperture 406 is a height H from the longitudinal axis of the post 402. In certain embodiments the post 402 is couplable with the drill sleeve 300. In at least one embodiment, the post 402 is integral with the drill sleeve 300 (e.g., see FIGS. 61A-63C). The sight aperture 406, in various embodiments, can be configured to hold a k-wire or other instrument to be a sight wire 408. The height H can be any height that places the sight wire 408 above a patient's foot, typically in the range of about five millimeters to about one hundred millimeters (5 mm-100 mm), among other heights that are less than 5 mm or greater than 100 m, each of which is contemplated herein. In other embodiments, the height H can be any height in the range of about ten millimeters to about forty millimeters, among other possible heights, each of which in contemplated herein. In further embodiments, the height H can be about twenty-five millimeters (25 mm), among other possible heights, each of which in contemplated herein.

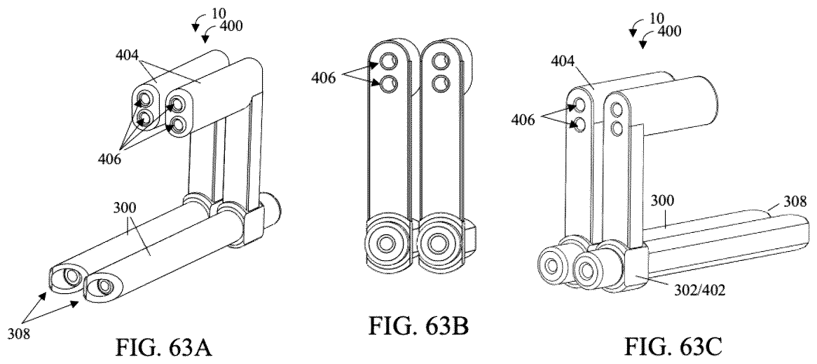

In one or more embodiments the surgical system 10 can include at least one AP radiograph positioning tool 410. In embodiments of the periscope 400 with at least two sight wires 408 (e.g., see FIGS. 63A-63C), the radiograph positioning tool 410 can be the two sight wires 408. When viewed on an AP radiograph, the two sight wires 408 will appear as one sight wire 408 indicating proper alignment.

In various embodiments the surgical system 10 can include a plurality of drill bits in various sizes, a plurality of screws 600 in various sizes, and a plurality of k-wires 276 in various sizes. The drill bits, and/or screws 600, and/or k-wires 276, and/or drill sleeve 300, and/or sight 404, and/or sight wire 408, and/or k-wire sheath 320 can be color-coded to indicate size (e.g., diameter). Color coding can help prevent mistakes during a surgical procedure, decrease time of the procedure, and improve patient outcomes.

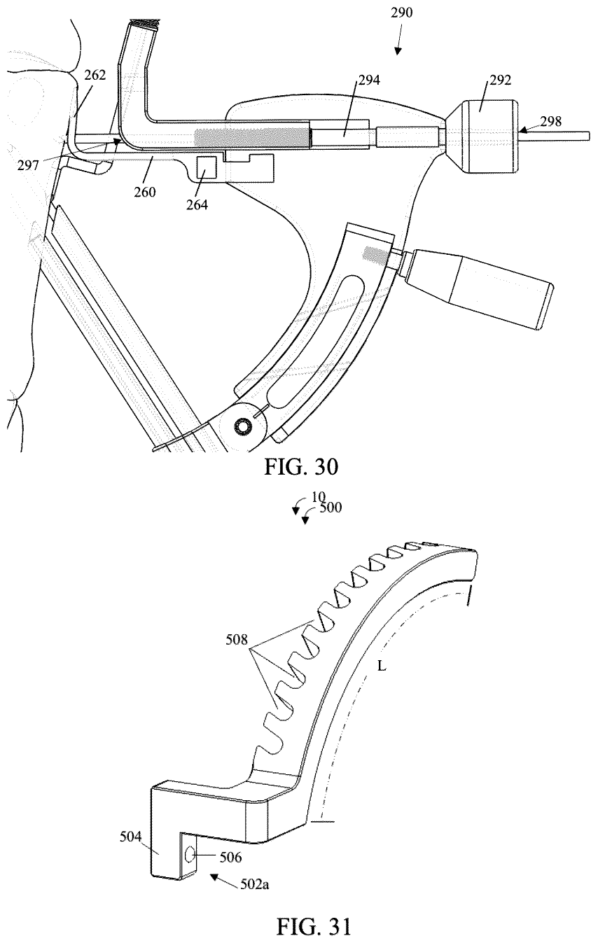

In some embodiments, the gadget 200 can include a translator 290 (e.g., see FIGS. 29-30). The translator 290 can be any mechanism that can move a bone and/or bone portion in a plane (e.g., the transverse plane, etc.).

The translator 290, in various embodiments, can include an activation mechanism 292 (see, e.g., FIG. 30), such as, for example, a dial, a slide, a ratchet, or any other activation means, each of which is contemplated herein. At least in the illustrated embodiment, the translator 290 can include a dial 292 coupled to a rod 294 such that in response to the dial 292 being turned a first direction (e.g., clockwise), the rod 294 turns a first direction, and in response to the dial 292 being turned a second direction (e.g., counterclockwise), the rod 294 turns a second direction.

The rod 294, in various embodiments, is coupled to the leg 206 such that in response to the rod 294 being turned in the first direction, the leg 206 extends, and in response to the rod 294 being turned in the second direction, the leg 206 retracts. In some embodiments, the translator 290 can be cannulated (e.g. see, aperture 297 and/or 298).

The gadget 200, in various embodiments, can include a buttress 260 that can include at least one prong 262 or other device and/or contour that can dissipate a force over a surface (see, e.g., FIGS. 30, 53, and 58-60B). The buttress 260 can include an aperture 264. In some embodiments, the buttress 260 includes two prongs 262 spaced apart from one another. The prongs 262, in various embodiments, are configured to apply force to a bone and/or bone portion.

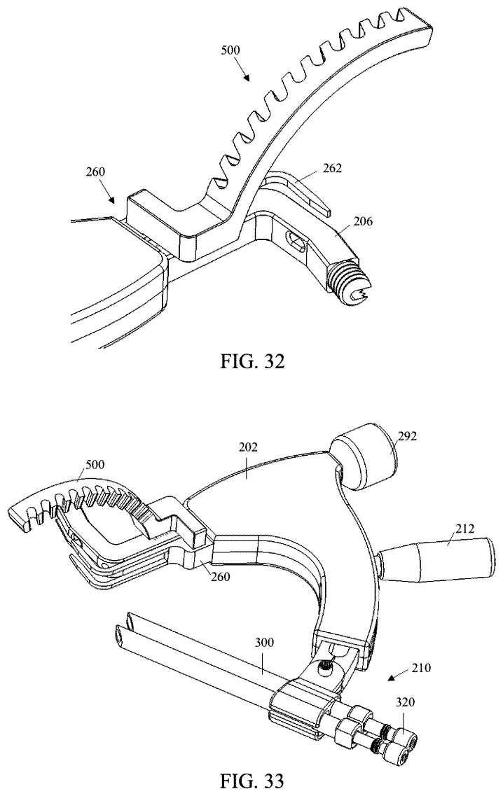

Now referring to FIGS. 31 through 33, in some embodiments, the surgical system 10 can include a rotation arm 500. The rotation arm 500, in various embodiments, can be coupled to the gadget 200 through an attachment mechanism 502, which can include attachment mechanism(s) 502a, 502b. In other embodiments the rotation arm 500 can be integral or permanently attached to the gadget 200.

The attachment mechanism 502a can include at least one of a set of threads, a post, an aperture, a shape, a detent, a ball, and/or a socket, or any other removably coupling means, each of which is contemplated herein. At least in the illustrated embodiment, the attachment mechanism 502a can include a post 504, and the attachment mechanism 502b can include an aperture 264 (e.g., see FIG. 16). The attachment mechanism 502a can include a ball 506, and the attachment mechanism 502b can include a socket.

The rotation arm 500, in certain embodiments, can be coupled to any portion of the gadget 200 that remains stationary relative to the buttress 260, and/or gadget 200, and/or leg 206, and/or body 202. In at least one embodiment, the rotation arm 500 is coupled to the gadget 200 in response to placing the post 504 of a first shape into the aperture 264 of a complimentary shape. In some embodiments, the shape is non-circular, which can limit and/or eliminate rotation of the post 504 within the aperture 264.

The post 504 shape can include any suitable shape. In various embodiments, the shape of the post 504 includes at least one of square, hexagonal, octagonal, hourglass, cloverleaf, cross, star, oval, and/or wedge, etc., among other shapes that are possible, each of which is contemplated herein. In other embodiments, the post 504 includes a circular shape, and other means of controlling the rotation of the post 504 within the aperture 264 are employed.

The rotation arm 500, in various embodiments, can include at least one set of slots or notches 508. The notches 508 can be evenly spaced along a length L of the rotation arm 500, or they can be unevenly spaced along the length L of the rotation arm 500. In at least the illustrated embodiment, the notches 508 are evenly spaced to create approximately five degrees (5°) of rotation for each notch 508. In at least one embodiment, the rotation arm is curved following a circular trajectory. In other embodiments, the rotation arm is curved following a non-circular trajectory.

In some embodiments, the notches can be spaced further apart to create degree changes of greater than 5°. In other embodiments, the notches can be spaced closer together to create degree changes of less than 5°. In further embodiments, the notches can be unevenly spaced further apart to create some degree changes of greater than 5° and closer together to create some degree changes of less than 5°.

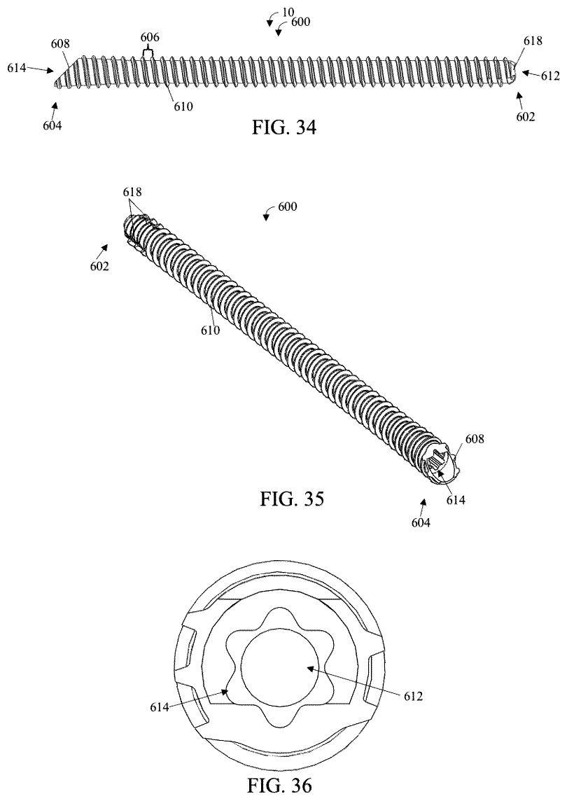

Now referring to FIGS. 34 through 36, in some embodiments, the surgical system 10 can include at least one screw 600. The screw 600, in various embodiments, can be fully threaded, or partially threaded.

In some embodiments, the screw 600 can be threaded at the first end 602. In additional or alternative embodiments, the screw 600 can be threaded at the second end 604. In certain embodiments, the screw 600 can include at least a portion of the shaft between the first end 602 and the second end 604, which can be non-threaded. In at least one embodiment, all the threads on screw 600 can include the same pitch 606 so as to not create compression or distraction as the screw 600 is inserted into bone portions (e.g., bones 704 and 706). In certain embodiments the threads on screw 600 can include different pitches to create compression and/or distraction as the screw 600 is inserted into bone portions.

In one or more embodiments, the second end 604 can include a chamfered shape 608. The chamfer shape 608 can be configured to conform to the shape of a bone (e.g., a proximal metatarsal 704).

In various embodiments, the screw 600 can include a longitudinal aperture 612 (e.g., the screw 600 can be canulated). Additionally, or alternatively, the aperture 612 can be circular so as to conform to the shape of a k-wire.

The aperture 612, in some embodiments, can be non-circular so as to conform to a different k-wire shape, or for other reasons. For example, the aperture 612 can be hexagonal, square, oval, or other shape(s), each of which is contemplated herein.

In some embodiments, the screw can have an aperture 614 that is configured to conform to a screwdriver, or other driving tool and/or similar tool. In one or more embodiments the aperture 612 and the aperture 614 are the same shape. In various embodiments aperture 612 and aperture 614 are different shapes. In various embodiments, the first end 602 can include a self-tapping and/or self-drilling geometry 618, which can eliminate the need for pre-tapping and/or pre-drilling.

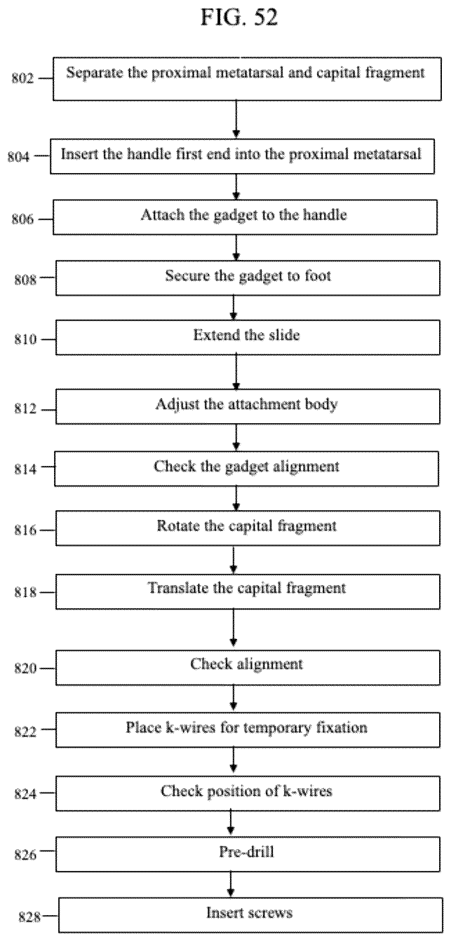

The following example procedure shown in FIG. 52 is of a bunion correction procedure, however, the surgical system 10 can be useful in other surgical procedures and/or other bones and/or bone portions. It is to be noted that the example procedure is intended as a guide to understanding the various embodiments disclosed herein and not to limit the various embodiments in any manner.

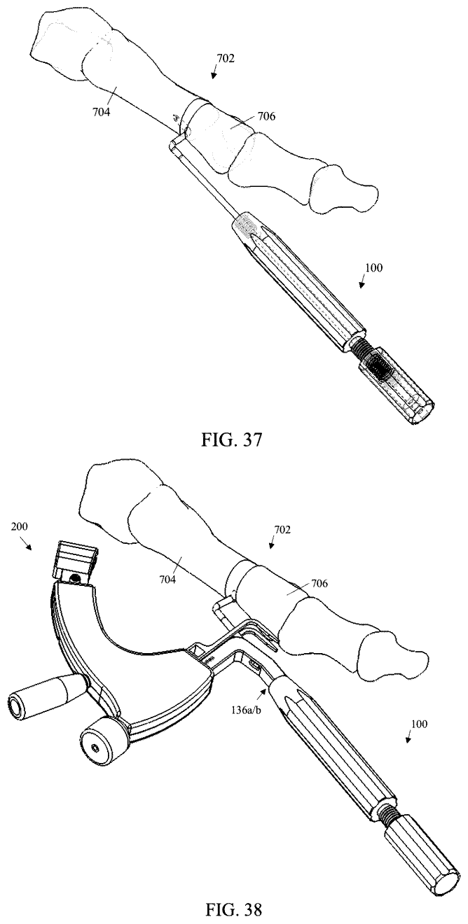

In FIG. 52, an osteotomy (or other procedure) can be performed on a metatarsal 702 to separate the capital fragment 706 from the proximal metatarsal 704 (block 802). In some embodiments, the capital fragment 706 may already be separated from the metatarsal 702, such as a fracture. The rod first end 104 can be placed within the proximal metatarsal 704, possibly within the medullary canal (block 804) (see, e.g., FIG. 37).

The gadget 200 can be coupled to the handle 100 by placing the slot 208 in the leg 206 over the rod 102, such that the rod 102 extends through the slot 208, and engaging the second attachment mechanism 136a/b. In at least one embodiment, the attachment mechanism 136a/b can be complimentary threads and the gadget 200 is coupled to the handle 100 by screwing the coupler first end 132 to the leg 206 (block 806) (see e.g., FIG. 38).

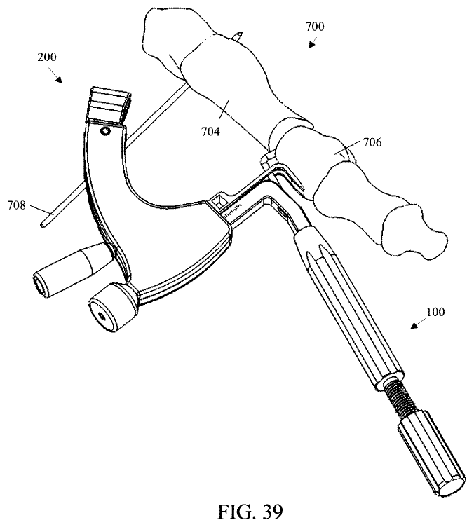

The gadget 200 can be secured to a foot 700 of a subject by engaging the body attachment mechanism 204. In at least one embodiment, engaging the body attachment mechanism 204 includes inserting a k-wire 708 through the body aperture 205 (block 808) (see, e.g., FIG. 39).

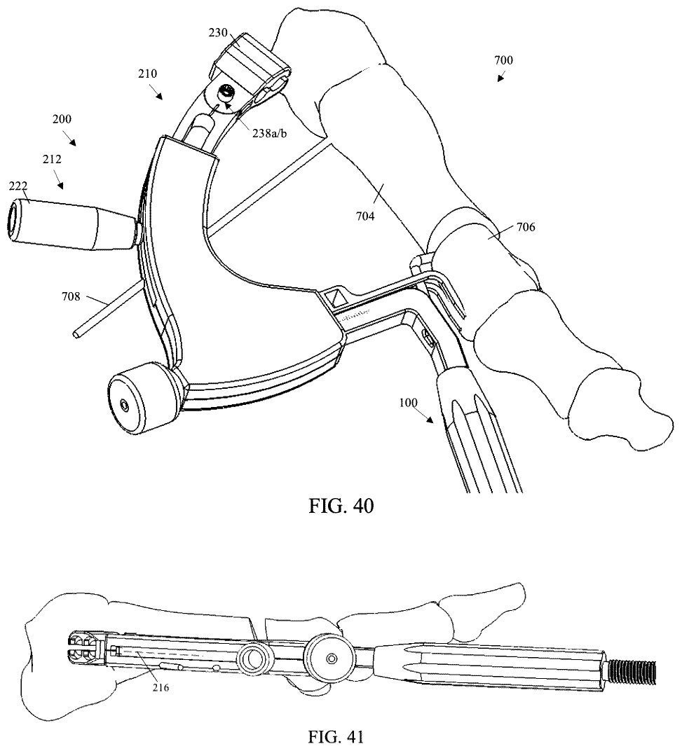

The slide 210 can be extended until it contacts the side of the foot 700 (see, e.g., FIG. 40) (block 810). The slide lock 212 is then engaged to keep the slide 210 from moving.

In some embodiments, the slide lock 212 can be engaged by rotating the lock handle 222 in a first direction (e.g., clockwise). The position of the guide body 230 can be evaluated.

If adjustment of the guide body 230 position is desired, the securing device 234 can be disengaged to allow for adjustment (block 812). In at least the illustrated embodiment, the securing device 234 includes the screw 238a and the aperture 238b. The securing device 234 can be disengaged by loosening the screw 238a, the position of the guide body 230 can be adjusted (i.e. the angle 250 is increased or decreased), and the securing device 234 can be re-engaged by tightening the screw 238a.

The alignment of the gadget 200 with the foot 700 can be confirmed radiographically (block 814). In some embodiments, the radiograph positioning tool 214 includes two bars 216 placed at opposing sides of the slide 210, but at the same depth. When viewed transversely, the two bars 216 will appear to be one bar (because they are aligned, or in the same plane) and appear to align with the longitudinal axis of the metatarsal 702 (see, e.g., FIG. 41) (block 816).

The capital fragment 706 can be rotated. In some embodiments the capital fragment 706 can be rotated freehand. In certain embodiments the capital fragment 706 can be rotated by inserting a k-wire 510 into the capital fragment 706. The k-wire 510 can act as a lever for manual rotation, and/or the rotation arm 500 can be utilized. In one or more embodiments, the k-wire 510 is placed such that the capital fragment 706 is being rotated about the capital fragment longitudinal axis. In various embodiments, the longitudinal axis of the capital fragment 706 is the center of rotation for the rotation arm 500. In certain embodiments, the k-wire 510 can include a stopper near the tip of the k-wire (e.g., an olive pin), or other means of preventing the k-wire from progressing further into the capital fragment 706.

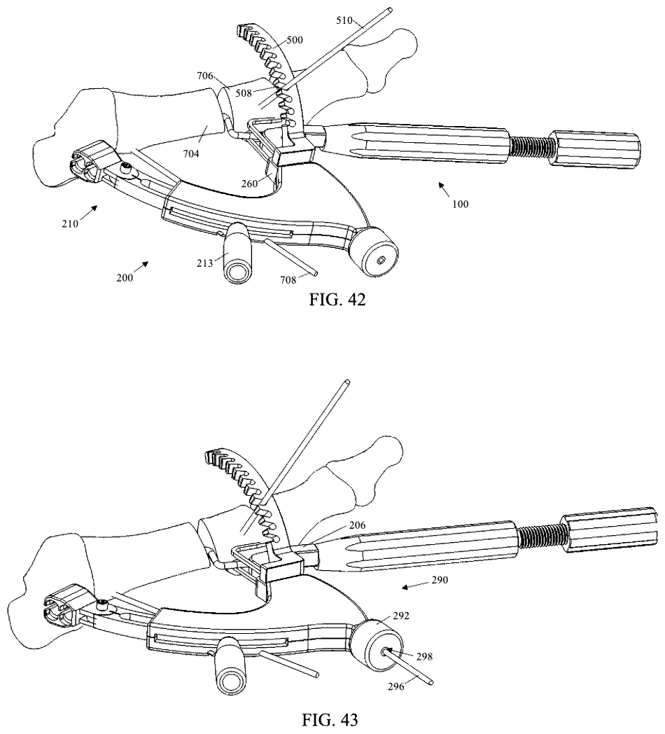

The rotation arm 500 can be coupled to the gadget 200 and/or any other portion of the surgical system 10 that remains stationary with respect to the capital fragment 706. In some embodiments, the rotation arm 500 can be coupled to the buttress 260 by placing the post 504 into the complimentary aperture 264, which can place the ball 506 in the socket 266. The k-wire 510 can be used as a lever to rotate the capital fragment 706 and then placed within a notch 508 to hold the rotation (see, e.g., FIG. 42).

A k-wire 296 can be placed through coextensive apertures 297 and 298 and into the capital fragment 706 (see, e.g., FIG. 43) to maintain the rotated position of the capital fragment 706. Optionally, the rotation arm 500 and the k-wire 510 can be removed. In some embodiments, the k-wire 296 can be inserted through aperture 297 prior to the gadget 200 being coupled to the rod 102. In certain embodiments, the k-wire 296 can include a stopper near the tip of the k-wire 296 (e.g., k-wire 296 can be an olive pin), or other means of preventing the k-wire from progressing further into the capital fragment 706.

The capital fragment 706 can be translated to the desired position (block 818). In one or more embodiments the capital fragment 706 can be moved transversely freehand. in various embodiments the capital fragment 706 can be moved transversely through the k-wire 296. In certain embodiments the capital fragment 706 can be moved transversely by utilizing the translator 290.

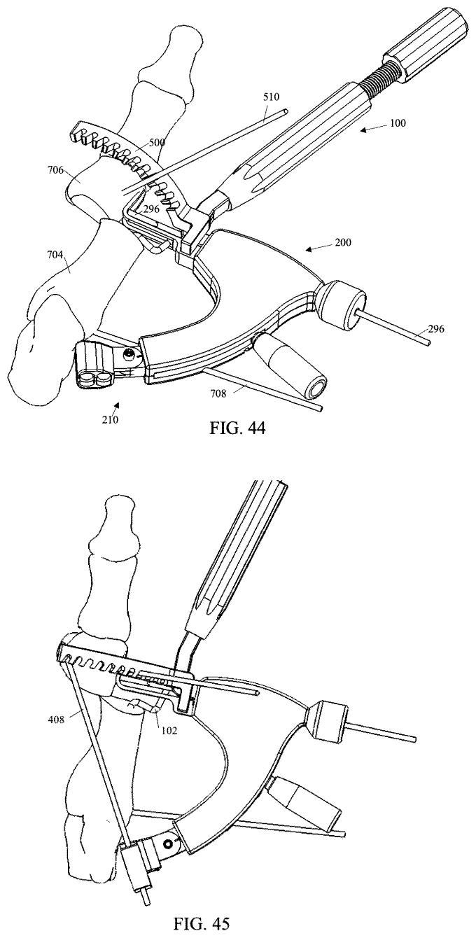

The translator 290 functions similarly to a screw jack. To utilize the translator 290, the dial 292 can be rotated to extend and/or retract the leg 206, placing force(s) between the proximal metatarsal 704 (through the rod first end 104) and the capital fragment (e.g., through the buttress 260, and/or k-wire 510, and/or k-wire 296), thus moving the capital fragment 706 in the transverse direction to a target and/or desired location (e.g., see FIGS. 44 and 45).

In some embodiments, the buttress 260 is configured to abut the skin of a subject. In other embodiments, the buttress 260 is configured to abut the capital fragment 706 of a subject (e.g., being placed between the skin and the capital fragment). In certain embodiments, the buttress 260 abuts or is adjacent to the capital fragment skin. Meaning that the buttress is near the capital fragment on the outside of the body. In at least one embodiment, the buttress is omitted.

In some embodiments, a periscope 400 can be employed to visualize screw placement (block 820). In various embodiments, a periscope post 402 can be placed in the sleeve aperture 240 and a sight wire 408 can be placed in the sight aperture 406.

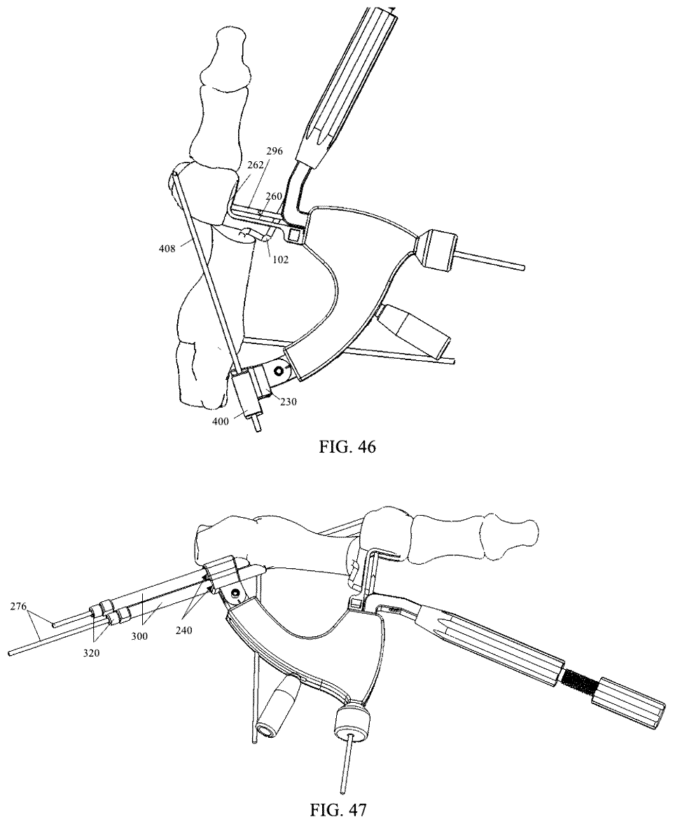

The sight wire 408 can extend over the foot 700. An AP radiograph can be taken, the future position/planned placement of screw 600 can be indicated by the sight wire 408 on the radiograph. The future position of screw 600 can be altered by adjusting the guide body 230 (see, block 812) and the slide 210 (see, block 810) (see, e.g., FIG. 45). If the rotation arm 500 is in the way of viewing the future position of the screw 600, it can be removed (see, e.g., FIG. 46). In some embodiments, the periscope can include a radiograph positioning tool 214 to assist in proper visualization.

The drill sleeve 300 and the guide wire sheath 320 can be placed in the sleeve aperture 240, and guide wire(s) 276 (e.g., k-wires) can be placed through the sheath(s) 320 into the proximal metatarsal 704 and into the capital fragment 706 (see, e.g., FIG. 47) (block 822). The position of the k-wires can be checked radiographically (block 824).

In various embodiments, the guide wire sheath 320 can be removed from the drill sleeve 300. In some embodiments, the sheath 320 can be removed by unscrewing the sheath 320 from the sleeve 300 (block 826). The bone(s) can be prepped by pre-drilling (e.g., using the drill sleeve 300 to guide the drill (see, e.g., FIG. 48)). In other embodiments the drill is canulated and the bone is pre-drilled over the guide wire(s) 276.

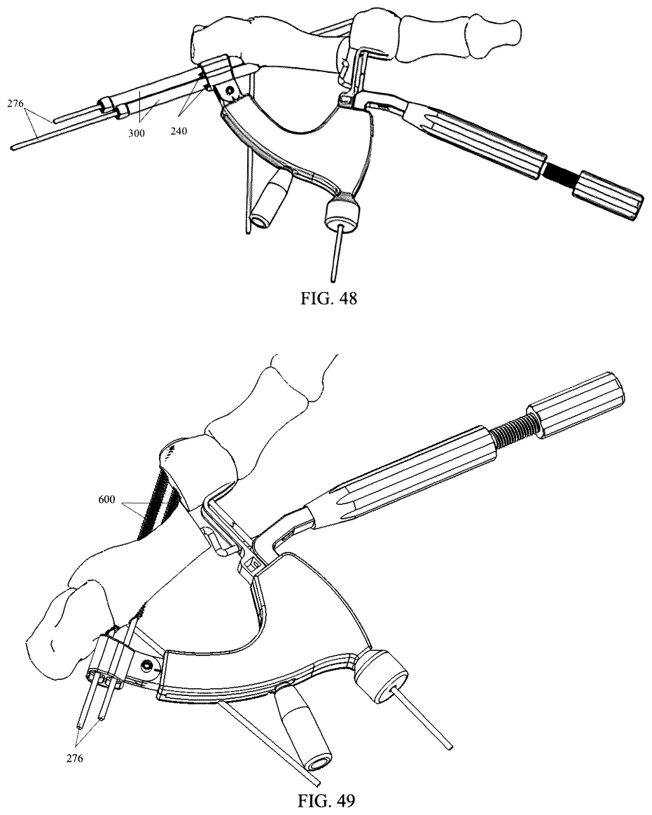

The drill sleeve 300 can be removed from the sleeve aperture 240 (block 828). The screw 600 can be placed over the guide wire 276. The screw 600 is placed through the proximal metatarsal 704 and into the capital fragment 706 (see, e.g., FIG. 49).

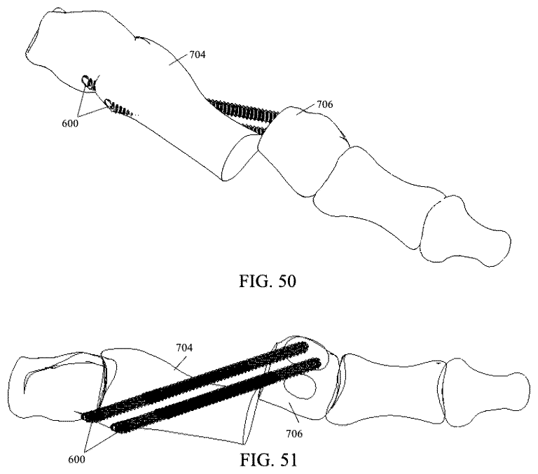

The screw 600 can be placed so that the chamfer 608 approximately aligns with the proximal metatarsal 704 edge. Aside from the screw(s) 600, the rest of the surgical system 10 can be removed (see, e.g., FIGS. 50 and 51).

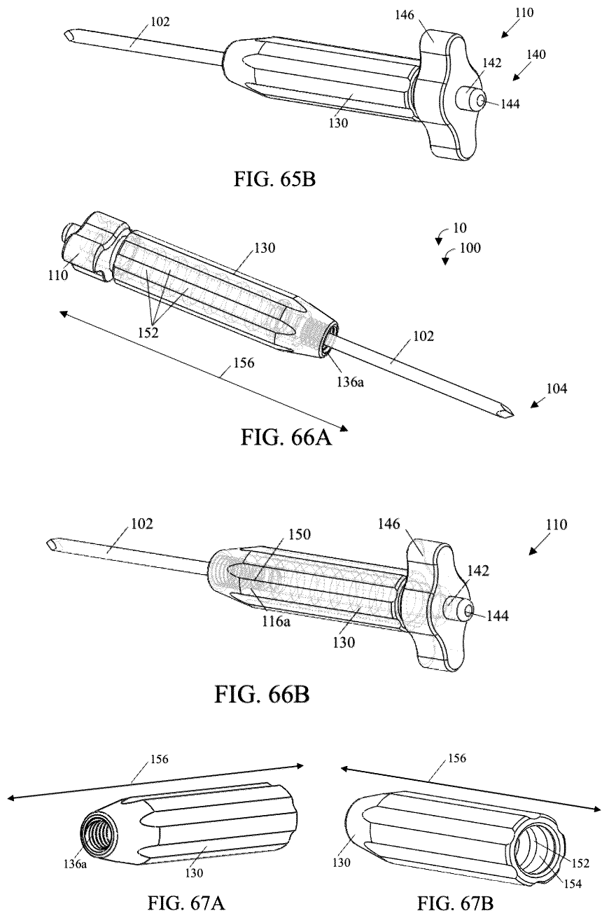

Referring now to FIGS. 53-66B. In one or more embodiments, the slot 208 through the leg 206 can be an open slot 209. The open slot 209 can be a passage with two sides open. The open slot 209 can allow for more maneuverability and ease of positioning the gadget 200 over the rod 102 (see, e.g., FIGS. 53-56).

In various embodiments, the buttress 260 can include a plate 268. The plate 268 can be in addition to, or instead of, the at least one prong 262. The plate 268 can include an inferior surface 272. In certain embodiments the inferior surface 272 can be flat. In other embodiments the inferior surface 272 can be non-flat (e. g, convex). In various embodiments the inferior surface 272 can include flat portions and non-flat portions. In certain embodiments the inferior surface 272 can be shaped to conform to a bone. In at least one embodiment the inferior surface 272 can be shaped to conform to a metatarsal.

In one or more embodiments, the buttress can include a cuff 280 (see, e.g., FIGS. 60A-B). The cuff 280 can include an inferior surface 282. In certain embodiments the inferior surface 282 can be flat. In other embodiments the inferior surface 282 can be non-flat (e. g, convex). In various embodiments the inferior surface 282 can include flat portions and non-flat portions. In certain embodiments the inferior surface 282 can be shaped to conform to a bone. In at least one embodiment the inferior surface 282 can be shaped to conform to a metatarsal. The cuff 280 can include a hole 284. The hole 284 can allow for the passage of a k-wire, such as k-wire 296, a k-wire other than k-wire 296, or other similar tool, each of which is contemplated herein. The cuff 280 may be symmetrical or asymmetrical in shape. In certain embodiments the cuff is rigid relative to the leg 206. In several embodiments the cuff 280 is rotatable relative to the leg 206.

In some embodiments, the attachment mechanism 116 can include a ball 150 and groove 152 (see, e.g., FIGS. 63-66). In certain embodiments the head 110 can include an actuator 140 that controls the movement of the ball 150 relative to the barrel 148. For example, the actuator 140 can control how proud the ball 150 is relative to the barrel 148. The actuator can be at least one: magnet, ball, groove, spring, thread, washer, bolt, screw, post, and/or hole, among other possible devices, each of which is contemplated herein. In many embodiments the actuator 140 can include a plunger 142. The plunger 142 can include a thumb rest 144. In at least one embodiment the head 110 can include a flange 146.

In certain embodiments the coupler 130 can include one or more groove(s) 152. In many embodiments the groove(s) 152 can be along an interior surface 154 of the coupler 130. In at least one embodiment, the groove(s) 152 can be configured to conform to ball 150. The groove(s) 152 can be circumferential, helical, and/or angled among other possible configurations that are possible, each of which is contemplated herein. In many embodiments the pitch of the helical groove can match the pitch of the threads of the second attachment mechanism 136a (such that as the coupler is screwed onto the leg, the amount of the rod 102 that is in the bone does not change).

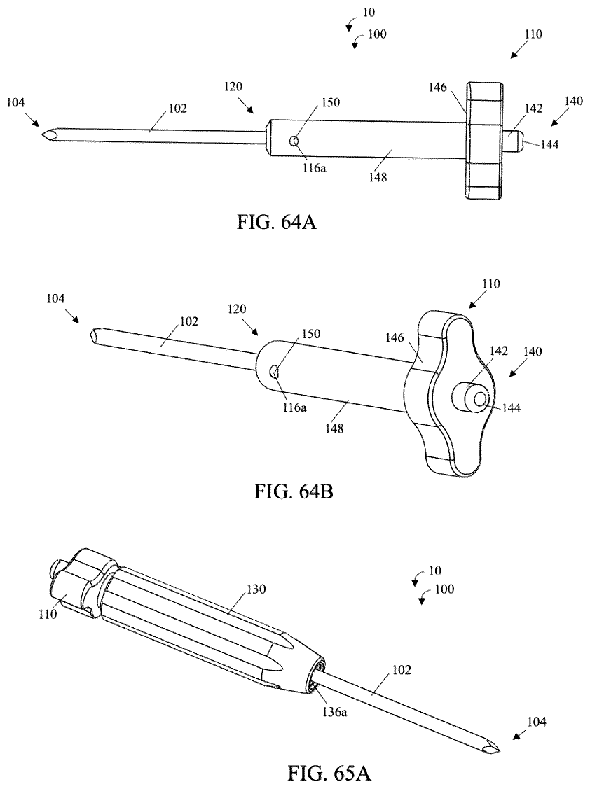

The gadget 200 position relative to rod 102 can be adjusted by altering the attachment mechanism 116. For example, if attachment mechanism 116 is complimentary threads (see, e.g., FIGS. 1 and 2), the position can be adjusted by screwing or unscrewing the coupler 130 and head 110. As another example, if the attachment mechanism 116 is ball 150 and groove(s) 152 (see, e.g., FIGS. 64 and 65), the position can be adjusted by engaging the actuator 140 to allow the ball 150 to recede within the coupler 130 (i.e. make ball 150 less proud relative to the barrel 148) to allow the coupler to be moved along the rod 102 to a desired location at which point the actuator 140 can be disengaged to allow the ball 150 to advance (i.e. become more proud relative to the barrel 148) within the groove 152. Ball 150 within groove 152 prevents the barrel 148 from moving in a longitudinal direction 156 relative to the coupler. In certain embodiments, the flange 146 can allow a surgeon to apply counter pressure when pressing the thumb rest 144 to engage the actuator 140, similar to a typical technique for using a syringe.

The various embodiments discussed herein may be practiced in other specific forms and the described embodiments are to be considered in all respects only as illustrative, and not restrictive. The scope of the technology is, therefore, indicated by the appended claims rather than by the foregoing description. All changes that come within the meaning and range of equivalency of the claims are to be embraced within their scope. That is, one of ordinary skill in the art will appreciate that modifications and/or adaptations to the various aspects may be made without departing from the scope of the present technology, as set forth in the following claims.

What is claimed is:

1. A surgical method comprising:
providing a surgical system comprising a handle, the handle comprising a rod and a coupler;
inserting a rod first end into a proximal metatarsal;
the surgical system further comprising a gadget, the gadget comprising:
a body;
a leg coupled to the body, the leg comprising a slot including at least one open side;
a slide slidably coupled to the body; and
a guide body coupled to the slide and configured to position a guide wire, wherein the guide body is rotatable relative to the slide;
after inserting the handle first end into the proximal metatarsal, coupling the gadget to the handle by placing the slot around the rod and engaging the attachment mechanism between the coupler and the leg;
extending the slide from a first slide position to a second slide position;
translating a capital fragment from a first bone position to a second bone position;
inserting at least one guide wire through the proximal metatarsal and the capital fragment.

2. The surgical method of claim 1, further comprising rotating the capital fragment from a third bone position to a fourth bone position.

3. The surgical method of claim 2, further comprising rotating the capital fragment before translating the capital fragment.

4. The surgical method of claim 2, further comprising rotating the capital fragment after translating the capital fragment.

5. The surgical method of claim 1, further comprising inserting at least one screw onto the guide wire and into the capital fragment, wherein the surgical system further comprises the at least one screw.

6. The surgical method of claim 5, further comprising inserting at least one screw onto the guide wire and into the capital fragment after removing the gadget and handle.

7. The surgical method of claim 5, further comprising inserting at least one screw onto the guide wire and into the capital fragment before removing the gadget and handle.

8. The surgical method of claim 1, wherein the coupler further comprises threads at a first end and the leg comprises threads, wherein the coupler threads and the leg threads are complimentary and coupling the gadget to the handle comprises screwing the coupler onto the leg threads.

9. The surgical method of claim 1, further comprising coupling the gadget to the proximal metatarsal by inserting a first k-wire through at least one body aperture and into the proximal metatarsal, wherein the body comprises at least one body aperture.

10. The surgical method of claim 1, further comprising engaging the slide lock to prevent the slide from moving relative to the body, wherein the slide comprises a slide lock.

11. The surgical method of claim 9, further comprising checking the alignment of the gadget with respect to the proximal metatarsal by:
viewing a lateral radiograph of the of the slide and the proximal metatarsal, wherein the slide comprises two bars on opposing sides of the slide that will appear as one bar along the proximal metatarsal longitudinal axis when the gadget is aligned with respect to the proximal metatarsal.

12. The surgical method of claim 2, wherein:
the surgical system further comprising a rotation arm coupled to the body, the rotation arm comprising at least one notch;
the surgical system further comprising a second k-wire;
the method further comprising inserting the second k-wire into the capital fragment;
using the second k-wire as a lever to rotate the capital fragment from the third bone position to the fourth bone position;
placing the second k-wire into the notch.

13. The surgical method of claim 1, the surgical system further comprising a translator comprising a passage in the leg and a post, wherein the passage and the post include complimentary threads;
wherein translating the capital fragment further comprises rotating the post in a first direction to control the extension of the leg, and rotating the post in a second direction to control the retraction of the leg.

14. The surgical method of claim 1, further comprising:
the surgical system further comprising at least one periscope, each periscope comprising a sight body coupled to a drill sleeve, wherein the sight body comprises a sight aperture;
inserting at least one drill sleeve into at least one sleeve aperture, wherein the guide body comprises at least one sleeve aperture.

15. The surgical method of claim 14, further comprising:
rotating the guide body from a first guide body position to a second guide body position;
viewing an anterior radiograph of the sight k-wire, proximal metatarsal, and capital fragment;
engaging a securing device, wherein the guide body comprises the securing device, and the securing device comprises an aperture and a screw.

16. The surgical method of claim 15, wherein the drill sleeve is non-circular, and the sleeve aperture is non-circular.

17. The surgical method of claim 1, wherein the slot is an open slot including two open sides.

18. A surgical method comprising:
providing a surgical system comprising a handle comprising a rod and a coupler including threads;
inserting a rod first end into a proximal metatarsal;
the surgical system further comprising a gadget, the gadget comprising:
a body;
a leg coupled to the body, wherein the leg comprises a slot including at least one open side, and the leg comprises threads;
a slide slidably coupled to the body; and
a guide body coupled to the slide and configured to position a guide wire, wherein the guide body is rotatable relative to the slide;
after inserting the handle first end into the proximal metatarsal, coupling the gadget to the handle by placing the slot around the rod and screwing the coupler threads onto the leg threads;
translating a capital fragment from a first bone position to a second bone position;
inserting at least one guide wire through the proximal metatarsal and the capital fragment;
extending the slide from a first slide position to a second slide position;
locking the slide into the second slide position;
providing a drill sleeve coupled to a k-wire sheath;
coupling the drill sleeve to the guide body;
placing a guide wire through the k-wire sheath;
removing the gadget and the handle;
inserting at least one screw onto the guide wire, through the proximal metatarsal and into the capital fragment.

19. The surgical method of claim 18, further comprising
providing a rotation arm comprising at least one notch;
inserting a k-wire into the capital fragment;
rotating the capital fragment from a third bone position to a fourth bone position, using the k-wire for leverage; and
placing the k-wire into the notch.

20. The surgical method of claim 19, wherein
the guide body comprises a k-wire escape slot;
removing the gadget further comprises guiding the guide wire through the k-wire escape slot.
